(19) United States
(12) Patent Application Publication
(10) Pub. No.: US 2023/0162821 A1
(43) Pub. Date: May 25, 2023

(54) INTERPRETATION MANAGEMENT DEVICE, RECORDING MEDIUM, AND INTERPRETATION MANAGEMENT METHOD

(71) Applicant: Konica Minolta, Inc., Tokyo (JP)

(72) Inventors: Shinsuke KATSUHARA, Tokyo (JP); Ken SATOU, Tokyo (JP); Hiroshi TERAO, Tokyo (JP)

(21) Appl. No.: 18/055,155

(22) Filed: Nov. 14, 2022

(30) Foreign Application Priority Data
Nov. 22, 2021 (JP) 2021-189077

(51) Int. Cl. G16H 10/40; G16H 10/60

(52) U.S. Cl. CPC G16H 10/40; G16H 10/60

(57) ABSTRACT

An interpretation management device includes a hardware processor. The hardware processor acquires auto-generated findings obtained by computer processing on medical information, acquires first interpretation findings created by a user based on the medical information, acquires second interpretation findings created by a user based on the medical information, compares the auto-generated findings with at least one of the first interpretation findings and the second interpretation findings, presents to a user a result of comparison between the auto-generated findings and the at least one interpretation findings based on a predetermined workflow, and allows the predetermined workflow to be set based on a user operation.

### CROSS-REFERENCE TO RELATED APPLICATIONS

The entire disclosure of Japanese Patent Application No. 2021-189077 filed on Nov. 22, 2021 is incorporated herein by reference in its entirety.

### BACKGROUND

### Technological Field

The present invention relates to an interpretation management device, a recording medium, and an interpretation management method.

### Description of the Related Art

In recent years, with the development of artificial intelligence (AI) technology, attempts have been made to introduce AI analysis in the medical field and support the analysis and diagnosis of medical information such as image diagnosis, which were conventionally done by doctors, by using AI.

In the medical field, it is required to perform examinations and diagnoses appropriately and quickly so that the burden on doctors is reduced by making diagnoses more efficient and optimized.

The introduction of AI analysis is expected to contribute to the efficiency and optimization of such diagnoses.

For example, JP 2017-010577A discloses a device including: an acquirer that acquires auto-generated findings, which are generated by analyzing examination data (medical information) using AI, and interpretation findings input through an operation interface for the examination data; and an evaluator that acquires change information operation-input by the user for the interpretation findings after presenting the auto-generated findings to the user and evaluates either the auto-generated findings or the interpretation findings based on the change information.

### SUMMARY

However, in Japan, double interpretation is recommended for, for example, lung cancer screening.

For example, when the technique described in JP 2017-010577A is used in double interpretation, there may be a case in which, after the end of the first interpretation by the first interpretation doctor, the results of the auto-generated findings or the interpretation findings of the first interpretation doctor are evaluated by the second interpretation doctor.

However, in the case of the flow in which the second interpretation doctor views the auto-generated findings or the interpretation findings of the first interpretation doctor first and then performs the second interpretation, the second interpretation may be biased. For this reason, there is a risk that the quality of the interpretation cannot be guaranteed.

In the second interpretation, there may be a demand for efficient interpretation by making the second interpretation doctor preferentially interpret images that are likely to have been overlooked by the first interpretation doctor.

However, with the known methods, it is not possible to check the result of comparison between the interpretation findings of the first interpretation doctor and the auto-generated findings before the second interpretation. For this reason, there is a problem that it is not possible to adopt a flow in which images that are likely to be overlooked are preferentially interpreted.

As described above, for example, different medical facilities require different workflows for interpretation or the like. In this respect, it takes cost and time to develop and manufacture each device (interpretation management device, analysis device) for realizing a desired workflow.

The present invention has been made in view of the above problems in the prior art, and it is an object of the present invention to provide an interpretation management device, a program, and an interpretation management method capable of realizing a desired workflow in interpretation.

To achieve at least one of the above mentioned objections, according to an aspect of the present invention, an interpretation management device reflecting one aspect of the present invention includes a hardware processor. The hardware processor acquires auto-generated findings obtained by computer processing on medical information, acquires first interpretation findings created by a user based on the medical information, acquires second interpretation findings created by a user based on the medical information, compares the auto-generated findings with at least one of the first interpretation findings and the second interpretation findings, presents to a user a result of comparison between the auto-generated findings and the at least one interpretation findings based on a predetermined workflow, and allows the predetermined workflow to be set based on a user operation.

According to another aspect, an interpretation management device includes a hardware processor. The hardware processor acquires auto-generated findings obtained by computer processing on medical information, acquires interpretation findings created by a user based on the medical information, compares the auto-generated findings with the interpretation findings, presents to a user a result of comparison between the auto-generated findings and the interpretation findings based on a predetermined workflow, and allows the predetermined workflow to be set based on a user operation.

According to another aspect, a non-transitory recording medium storing a computer readable program causes a computer to perform: acquiring auto-generated findings obtained by computer processing on medical information; acquiring first interpretation findings created by a user based on the medical information; acquiring second interpretation findings created by a user based on the medical information; comparing the auto-generated findings with at least one of the first interpretation findings and the second interpretation findings; presenting to a user a result of comparison between the auto-generated findings and the at least one interpretation findings based on a predetermined workflow; and allowing setting of the predetermined workflow based on a user operation.

According to another aspect, a non-transitory recording medium storing a computer readable program causing a computer to perform: acquiring auto-generated findings obtained by computer processing on medical information; acquiring interpretation findings created by a user based on the medical information; comparing the auto-generated findings with the interpretation findings; presenting to a user a result of comparison between the auto-generated findings and the interpretation findings based on a predetermined workflow; and allowing setting of the predetermined workflow based on a user operation.

According to another aspect, an interpretation management method includes: acquiring auto-generated findings obtained by computer processing on medical information; acquiring first interpretation findings created by a user based on the medical information; acquiring second interpretation findings created by a user based on the medical information; comparing the auto-generated findings with at least one of the first interpretation findings and the second interpretation findings; presenting to a user a result of comparison between the auto-generated findings and the at least one interpretation findings based on a predetermined workflow; and allowing setting of the predetermined workflow based on a user operation.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a diagram showing the overall configuration of a medical imaging system according to the present embodiment;

FIG. 2 is a block diagram of main parts showing the functional configuration of an embodiment of an analysis device as an interpretation management device according to the present invention;

FIG. 3 is a diagram showing an example of a worklist display screen;

FIG. 4 is a diagram showing an example of a workflow setting screen;

FIG. 5 is an explanatory diagram schematically showing the content of a “first workflow” set on the workflow setting screen shown in FIG. 4;

FIG. 6 is a diagram showing an example of a workflow setting screen;

FIG. 7 is an explanatory diagram schematically showing the content of a “second workflow” set on the workflow setting screen shown in FIG. 6;

FIG. 8 is a diagram showing an example of a workflow setting screen; and

FIG. 9 is an explanatory diagram schematically showing the content of a “third workflow” set on the workflow setting screen shown in FIG. 8.

### DETAILED DESCRIPTION OF THE EMBODIMENTS

Hereinafter, embodiments of an interpretation management device, an interpretation management method, and a recording medium according to the present invention will be described. However, the scope of the present invention is not limited to the illustrated examples.

[Configuration of Medical Imaging System]

The interpretation management device according to the present embodiment performs, for example, analysis of medical images that are medical information in a medical image system. An “analysis device” described below functions as the “interpretation management device”.

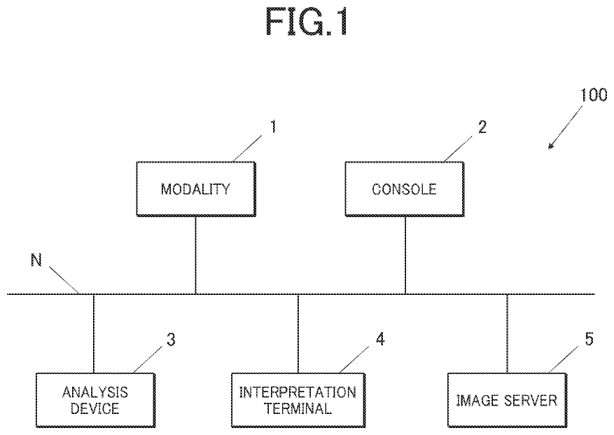

FIG. 1 shows the system configuration of a medical imaging system 100.

As shown in FIG. 1, the medical imaging system 100 includes a modality 1, a console 2, an analysis device 3, an interpretation terminal 4, an image server 5, and the like, and these are connected to each other through a communication network N, such as a local area network (LAN), a wide area network (WAN), and the Internet. Each device forming the medical imaging system 100 conforms to the health level seven (HL7) or digital image and communications in medicine (DICOM) standard, and communication between the devices is performed according to the HL7 or DICOM. The number of modalities 1, consoles 2, interpretation terminals 4, and the like is not particularly limited.

The modality 1 is an image generation device such as an X-ray imaging device (DR, CR), an ultrasonic diagnostic device (US), a CT, and an MRI, and generates a medical image as medical information by imaging a patients examination target site as a subject based on examination order information transmitted from a radiology information system (RIS; not shown). In a medical image generated by the modality 1, supplementary information (patient information, examination information, image ID, and the like) is written, for example, in the header of the image file according to the DICOM standard. The medical image attached with the supplementary information as described above is transmitted to the analysis device 3 or the interpretation terminal 4 through the console 2 or the like.

The console 2 is an imaging control device that controls imaging in the modality 1. The console 2 outputs imaging conditions or image reading conditions to the modality 1 and acquires image data of medical images captured by the modality 1. The console 2 includes a hardware processor, a display, an operation interface, a communicator, a storage, and the like (not shown), and these are connected to each other by a bus.

The analysis device 3 is a device that performs various analyses on a medical image, which is medical information, and is an interpretation management device in the present embodiment. The analysis device 3 is configured as a PC, a mobile terminal, or a dedicated device. In the present embodiment, the analysis device 3 includes a medical image management device, such as a picture archiving and communication system (PACS).

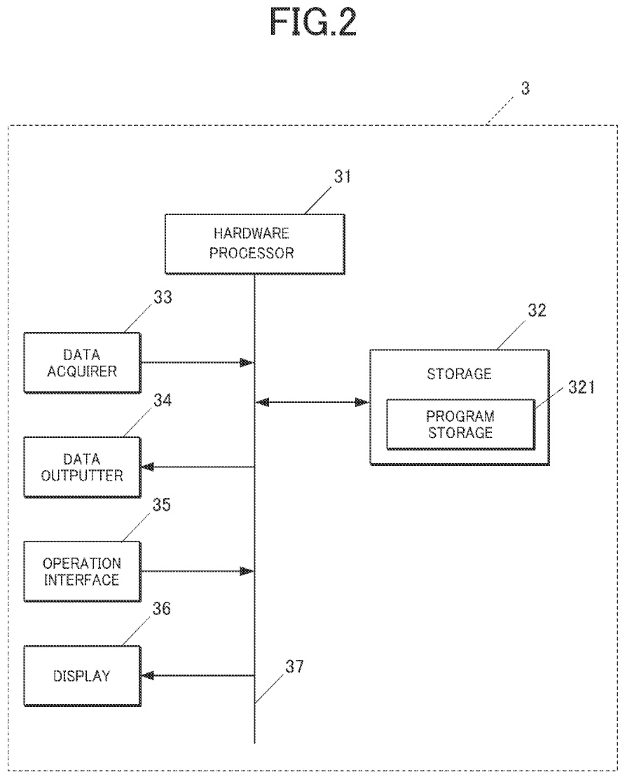

FIG. 2 is a block diagram showing the functional configuration of the analysis device 3.

As shown in FIG. 2, the analysis device 3 includes a hardware processor 31, a storage 32, a data acquirer 33, a data outputter 34, an operation interface 35, a display 36, and the like, and these are connected to each other through a bus 37.

The data acquirer 33 is an acquirer that acquires various kinds of data from an external device (for example, the console 2 or the interpretation terminal 4 described later).

The data acquirer 33 is, for example, a network interface, and is configured to receive data from an external device connected through the communication network N in a wired or wireless manner. In the present embodiment, the data acquirer 33 is a network interface, but can also be a port or the like into which a USB memory, an SD card, and the like can be inserted.

In the present embodiment, the data acquirer 33 acquires image data of a medical image from the console 2, for example. The data acquirer 33 acquires, from the interpretation terminal 4, a diagnosis result (lesion detection result information that can be read from the medical image) relevant to the medical image created by the user (for example, a doctor) based on the medical image, which is medical information, and information such as an interpretation report, which is the result of interpretation by an interpretation doctor (for example, interpretation doctors who perform first and second interpretations).

Specifically, the data acquirer 33 functions as a “second acquirer” that acquires “first interpretation findings” (result of first interpretation) created by the user (for example, a first interpretation doctor) based on medical information and a “third acquirer” that acquires a “second interpretation findings” (result of second interpretation) created by the user (for example, a second interpretation doctor) based on medical information. When there is supplementary information, such as when a region of interest (ROI) is set in a medical image by the user such as an interpretation doctor, the data acquirer 33 acquires such supplementary information.

The “first interpretation findings” and “second interpretation findings” acquired by the data acquirer 33 include various kinds of information, such as information regarding the presence or absence of a lesion (that is, information indicating whether the detection of an abnormal part is “+ (with abnormal findings)” or “− (no abnormal findings)”) and information of the name of the lesion or the location of the lesion. The specific content of the information included in the “first interpretation findings” and “second interpretation findings” is not limited to those exemplified herein, and may be a part of these or may include information other than these.

The “first interpretation findings” or the “second interpretation findings” acquired by the data acquirer 33 are transmitted to the hardware processor 31.

The data outputter 34 is an outputter that outputs information processed by the analysis device 3. The destination to which the data outputter 34 outputs various kinds of information is not particularly limited. For example, the destination to which the data outputter 34 outputs various kinds of information may be the display 36 of the analysis device 3, or may be the interpretation terminal 4 or the image server 5 to be described later or various external display device (not shown).

As will be described later, in the present embodiment, a “predetermined workflow” is set based on a user operation, and the information processed by the analysis device 3 is output to the display 36 or the like so that various displays are performed based on the set “predetermined workflow”.

As the data outputter 34, for example, ports of various media such as a network interface for communicating with the interpretation terminal 4, the image server 5, and the like, a connector for connecting to an external device (for example, a display device or a printer (not shown)), and a USB memory can be applied.

The operation interface 35 is, for example, a keyboard including various keys, a pointing device such as a mouse, or a touch panel attached to the display 36. The operation interface 35 can be operated for input by the user. Specifically, the operation interface 35 outputs an operation signal input by a key operation on a keyboard, a mouse operation, or a touch operation on a touch panel to the hardware processor 31.

In the present embodiment, as will be described later, the user can customize a desired workflow (specific procedures for interpretation and the like), and the operation interface 35 receives the user's input operation and outputs an operation signal according to the input to the hardware processor 31.

The display 36 includes a monitor, such as a liquid crystal display (LCD), and displays various screens according to instructions of a display signal input from the hardware processor 31. The number of monitors is not limited to one, and a plurality of monitors may be provided.

As will be described later, the display 36 appropriately displays various kinds of information based on display data output from the hardware processor 31.

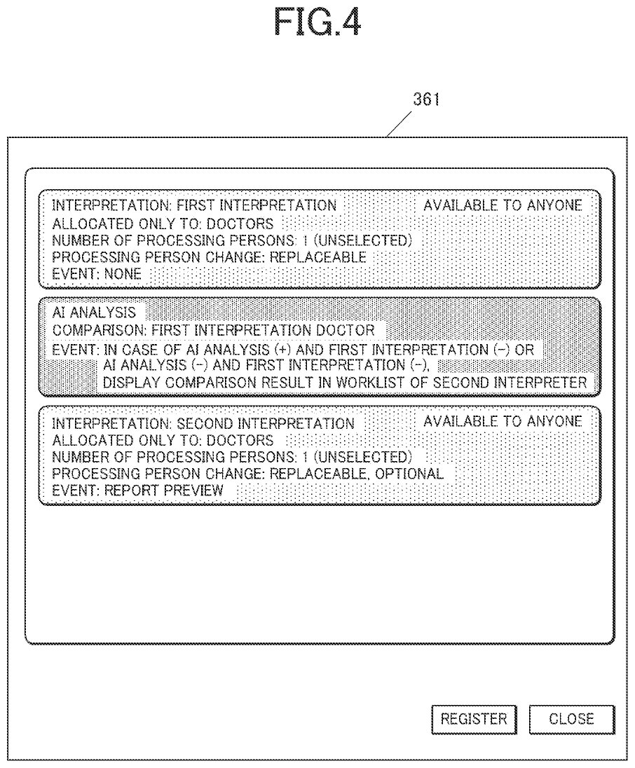

In the present embodiment, the user can customize a desired workflow (specific procedures for interpretation and the like) as described above, and a workflow setting screen 361 (see FIGS. 4, 6, and 8 as an example of the workflow setting screen 361) for inputting and setting the “predetermined workflow” is displayed on the display 36. In order to achieve the desired flow, the user can set the order of interpretation (first interpretation or second interpretation) and AI analysis comparison processing, conditions for performing various kinds of processing, the content of specific processing performed in various kinds of processing, and the like while viewing the screen by operating the operation interface 35 or the like.

A touch panel may be integrated with the display screen of the display 36. In this case, the user can perform various inputs, such as replacing the frame in the workflow setting screen 361 and rewriting the descriptions in each frame, by touch operation.

In the present embodiment, the display 36 functions as a presenter that presents a result of comparison between the “auto-generated findings”, which will be described later, and at least one interpretation findings (that is, “first interpretation findings” or “second interpretation findings”) to the user based on the “predetermined workflow”.

The content displayed on the display 36 is not limited to the result of comparison between the “auto-generated findings” and the interpretation findings, and various kinds of information or images can be displayed.

Those functioning as presenters are not limited to the display 36, and may be, for example, the interpretation terminal 4, the image server 5, or various external display devices.

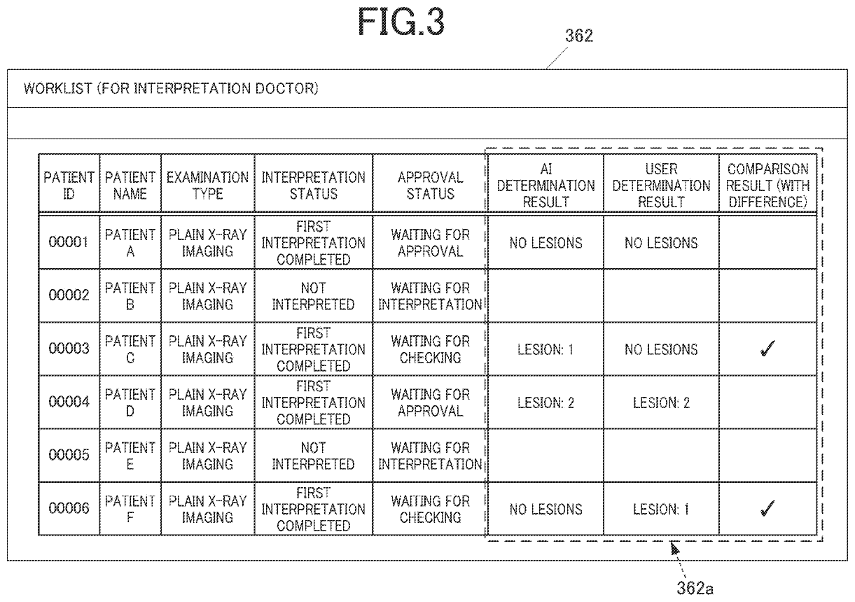

FIG. 3 is a diagram showing an example of a worklist display screen 362 including a result of comparison between the “auto-generated findings” and the interpretation findings displayed on the presenter (display 36 or the like).

The worklist display screen 362 is a screen that doctors (the first interpretation doctor and the second interpretation doctor) who are users refer to when performing interpretation (first interpretation, second interpretation, checking interpretation, and the like).

As shown in FIG. 3, on the worklist display screen 362, the interpretation schedule is displayed so as to be associated with “patient ID”, “patient name”, “examination type”, and the like. “Interpretation status” and “approval status” are displayed so that the person viewing the screen can easily understand the current stage of the examination of the patient on the list.

“Auto-generated findings” (referred to as an “AI determination result” in FIG. 3) that are the result of AI analysis to be described later, interpretation findings (that is, “first interpretation findings” or “second interpretation findings”; referred to as a “user determination result” in FIG. 3) of a doctor (first interpretation doctor, second interpretation doctor, or the like) who is the user, a result of comparison between the “auto-generated findings” and the interpretation findings (at least one of the “first interpretation findings” and the “second interpretation findings”), and the like are displayed on the worklist display screen 362. In the example shown in FIG. 3, if there is a difference as a result of the comparison, a check mark is displayed.

In FIG. 3, a portion (in FIG. 3, a display portion of “AI determination result”, “user determination result”, and “comparison result (with difference)”) of the worklist display screen 362 is shown as a findings presentation column 362a so as to be surrounded by the dotted line.

The content displayed in the findings presentation column 362a in the worklist display screen 362 will be specifically described with reference to FIG. 3.

For example, in the illustrated example, in the case of a patient A, AI analysis and first interpretation of the first interpretation doctor are performed. The “auto-generated findings” (“AI determination result” in FIG. 3) obtained by the AI analysis are “no lesion” (that is, “no abnormality (−)”), the “first interpretation findings” (“user determination result” in FIG. 3) obtained as a result of the first interpretation are also “no lesion” (that is, “no abnormality (−)”), and the result of comparison between the “auto-generated findings” and the “first interpretation findings” is “match” (that is, no difference).

On the other hand, for example, in the case of a patient C, AI analysis and first interpretation of the first interpretation doctor are similarly performed. However, the “auto-generated findings” (“AI determination result” in FIG. 3) obtained by the AI analysis are “lesion: 1” (that is, “with abnormality (+)” in which one lesion is observed), the “first interpretation findings” (“user determination result” in FIG. 3) obtained as a result of the first interpretation are “no lesion” (that is, “no abnormality (−)”), and the result of comparison between the “auto-generated findings” and the “first interpretation findings” is “mismatch” (that is, with difference).

The hardware processor 31 includes a central processing unit (CPU), a random access memory (RAM), and the like, and performs overall control of the operation of each unit of the analysis device 3. Specifically, the CPU reads various processing programs stored in a program storage 321 of the storage 32, loads the processing programs to the RAM, and executes various kinds of processing according to the programs. In the present embodiment, the hardware processor 31 implements various functions as described below in cooperation with the programs.

For example, the hardware processor 31 functions as a “first acquirer” that acquires “auto-generated findings” obtained by computer processing on medical information.

Specifically, lesion detection and analysis processing is performed on the medical image acquired by the data acquirer 33, and one or more types of lesion detection and analysis results are output as “first medical information.” As the computer processing, for example, AI analysis using artificial intelligence (AI) for image diagnosis and image analysis including detection of lesions by computer aided diagnosis (CAD) are used.

In the present embodiment, the case is exemplified in which the hardware processor 31 as a “first acquirer” acquires, as the “auto-generated findings”, the detection result of the presence or absence of a lesion (that is, the result indicating that the detection of an abnormal part is “+ (with abnormal findings)” or “− (no abnormal findings)”). However, the “auto-generated findings” are not limited to the detection result of the presence or absence of a lesion. For example, the hardware processor 31 may function as a learner (not shown) that learns the correspondence between medical information (medical image in the present embodiment) and medical information (lesion name and the like), and may obtain “auto-generated findings” by computer processing on the medical information (medical image) based on the learned correspondence between the medical information (medical image) and the medical information.

That is, for example, by using a machine learning model created by learning such as deep learning using a large amount of training data (a pair of a medical image showing a lesion and a correct answer label (a lesion area in the medical image, the diagnosis name of the lesion (type of lesion), and the like)), the lesion is detected and analyzed from the input medical image.

When the “auto-generated findings” are acquired as described above, information such as the name of the lesion or the location of the lesion is attached to the image data of the medical image as supplementary information.

In the present embodiment, the hardware processor 31 also functions as a comparer.

The hardware processor 31 as a comparer compares the “auto-generated findings” acquired by the hardware processor 31 as a “first acquirer” with at least one of the “first interpretation findings” and the “second interpretation findings” acquired through the data acquirer 33.

The hardware processor 31 as a comparer compares information regarding the presence or absence of abnormal findings extracted from the “auto-generated findings” with information regarding the presence or absence of abnormal findings extracted from at least one interpretation findings (that is, “first interpretation findings” or “second interpretation findings”), and derives (calculates) a result of the comparison (comparison result).

Specifically, match or mismatch (difference) between the information of “auto-generated findings” and the information of the interpretation findings is clarified.

For example, when information indicating that an abnormal part is detected (that is, “+” indicating that there are abnormal findings) is extracted from the “auto-generated findings” that are the result of AI analysis and information indicating that an abnormal part is detected (that is, “+” indicating that there are abnormal findings) is also extracted from the interpretation findings (that is, the “first interpretation findings” or the “second interpretation findings”), both the “auto-generated findings” by AI and the interpretation findings of the doctor are “+” (with abnormal findings) information. Therefore, the hardware processor 31 as a comparer derives (calculates) a comparison result that both the results match each other as “+” (with abnormal findings).

On the other hand, for example, when information indicating that an abnormal part is detected (that is, “+” indicating that there are abnormal findings) is extracted from the “auto-generated findings” that are the result of AI analysis and information indicating that no abnormal part is detected (that is, “−” indicating that there are no abnormal findings) is extracted from the interpretation findings (that is, the “first interpretation findings” or the “second interpretation findings”), the hardware processor 31 as a comparer derives (calculates) a comparison result that the “auto-generated findings” by AI analysis “+” (with abnormal findings) and the interpretation result of the doctor “−” (no abnormal findings) do not match (are different).

In the present embodiment, the information extracted from the “auto-generated findings” can be “+” information indicating that there are abnormal findings and “−” information indicating that there are no abnormal findings.

The information extracted from the interpretation findings (that is, “first interpretation findings” or “second interpretation findings”) can also be “+” information indicating that there are abnormal findings and “−” information indicating that there are no abnormal findings.

The information extracted from the “auto-generated findings” or the interpretation findings (“first interpretation findings” or “second interpretation findings”) is not limited to the presence or absence of abnormal findings, and can be the types, number, locations, and the like of abnormal findings. In the following embodiment, the explanation will be given on the assumption that the hardware processor 31 as a comparer performs comparison processing on the presence or absence of abnormal findings.

“Auto-generated findings” are structured data obtained by computer processing (AI analysis). On the other hand, the interpretation findings (“first interpretation findings” or “second interpretation findings”) are not limited to structured ones, such as an interpretation report created by a doctor.

For example, the presence or absence of abnormal findings may be expressed as “+” or “−”, or may be expressed by a character string indicating the presence or absence of an abnormality, such as “there is an abnormality” or “there is no abnormality” or “abnormality is recognized” or “abnormality is not recognized”. There may be variations in expression. For this reason, on the assumption that the hardware processor 31 as a comparer compares the “auto-generated findings” with the interpretation findings (“first interpretation findings” or “second interpretation findings”), the hardware processor 31 may have a function of structuring the interpretation findings to obtain data (structured data) including character strings and the like that can be compared with the “auto-generated findings”. In this case, for example, dictionary data defining the correspondence relationship between character strings, which is used for generating structured data, is stored in the storage 32, and the hardware processor 31 structures the interpretation findings with reference to the dictionary data.

The hardware processor 31 broadly defines various expressions or terms in the interpretation findings. For example, when expressions such as “+”, “there is an abnormality”, and “abnormality is recognized” can be associated as the same meaning as “information indicating abnormal findings (+)” extracted from “auto-generated findings” and expressions such as “−”, “there is no abnormality”, and “abnormality is not recognized” can be associated as the same meaning as “information indicating no abnormal findings (−)” extracted from “auto-generated findings”, even if there are some discrepancies in the expression between the “auto-generated findings” and the interpretation findings, the hardware processor 31 as a comparer can determine match or mismatch between the “auto-generated findings” and the interpretation findings. In this case, comparison processing between the “auto-generated findings” and the interpretation findings (“first interpretation findings” or “second interpretation findings”) may be performed without structuring the interpretation findings.

In the present embodiment, the hardware processor 31 functions as a setter that enables the setting of a “predetermined workflow” based on the user operation.

In the present embodiment, the hardware processor 31 as a setter can set the “predetermined workflow” so as to be associated with the information of the “auto-generated findings” and the information of at least one interpretation findings (that is, “first interpretation findings” or “second interpretation findings”).

For example, when information indicating that there are abnormal findings (+) is extracted from the “auto-generated findings” and information indicating that there are no abnormal findings (−) is extracted from at least one interpretation findings (that is, “first interpretation findings” or “second interpretation findings”), the hardware processor 31 as a setter can set the “predetermined workflow” by the user operation.

The “predetermined workflow” referred to herein is the content of a procedure, such as “which doctor will interpret images in what order?” or “what kind of” information the doctor refers to (or does not refer to) when creating interpretation findings (that is, “first interpretation findings” or “second interpretation findings”).

The user can input a desired workflow by performing an input operation through the operation interface 35 or by touching the setting screen, and the hardware processor 31 as a setter receives such a user operation and sets a “predetermined workflow” based on the user operation.

When the hardware processor 31 as a setter sets the “predetermined workflow”, it is possible to set whether or not to display the “auto-generated findings” or at least one interpretation findings (that is, “first interpretation findings” or “second interpretation findings”) so as to be associated with the information (that is, information indicating that there are abnormal findings (+) or there are no abnormal findings (−)) of the “auto-generated findings” and the information (that is, information indicating that there are abnormal findings (+) or there are no abnormal findings (−)) of at least one interpretation findings (that is, “first interpretation findings” or “second interpretation findings”).

Specifically, as the “predetermined workflow”, for example, the hardware processor 31 as a setter in the present embodiment can set at least two workflows among a workflow in which the “second interpretation findings” are acquired after comparing the “auto-generated findings” and the “first interpretation findings” with each other (hereinafter, referred to as a “first workflow”), a workflow in which the “second interpretation findings” are acquired after acquiring the “first interpretation findings” and the “auto-generated findings” and the “second interpretation findings” are compared with each other (hereinafter, referred to as a “second workflow”), and a workflow in which the “auto-generated findings”, the “first interpretation findings”, and the “second interpretation findings” are compared with each other (hereinafter, referred to as a “third workflow”). The specific content, type, and the like of the “predetermined workflow” are not limited to those exemplified herein.

The storage 32 is a hard disc drive (HDD), a semiconductor memory, and the like, and includes the program storage 321 that stores programs for performing various kinds of processing including comparison processing on medical information, such as medical images, and workflow setting processing. The storage 32 also stores parameters, files, and the like necessary for executing the programs stored in the program storage 321.

As described above, in the hardware processor 31, when performing processing for generating structured data, which can be compared with the “auto-generated findings”, by structuring unstructured data such as an interpretation report created by the user (interpretation doctor), dictionary data (structural dictionary) and the like used to perform the structuring processing are also stored in the storage 32.

The interpretation terminal 4 is, for example, a computer device that includes a hardware processor, an operation interface, a display, a storage, a communicator, and the like and that reads a medical image, which is medical information, from the image server 5 or the like and displays the medical image for interpretation.

The user (first interpretation doctor, second interpretation doctor, and the like) interprets the medical image at the interpretation terminal 4 and creates an interpretation report or the like, which is a diagnosis result of the interpretation doctor regarding the medical image.

The image server 5 is, for example, a server forming a picture archiving and communication system (PACS), and stores each medical image output from the modality 1 in a database so that patient information (patient ID, patient name, date of birth, age, sex, height, weight, and the like), examination information (examination ID, examination date and time, modality type, examination site, requesting department, examination purpose, and the like), an image ID of the medical image, interpretation findings (that is, “first interpretation findings” or “second interpretation findings”) of the interpretation doctor such as information (that is, “auto-generated findings”) of AI analysis results output from the hardware processor 31 of the analysis device 3 and an interpretation report created by the user (interpretation doctor) at the interpretation terminal 4, a comparison result output from the hardware processor 31 (hardware processor 31 as a comparer) of the analysis device 3, and the like are associated with the medical image.

[Regarding Interpretation Management Method in Present Embodiment]

In the present embodiment, an interpretation management method includes: a first acquisition step for acquiring “auto-generated findings” obtained by computer processing (AI analysis in the present embodiment) on medical information, an acquisition step (“second acquisition step” or “third acquisition step”) for acquiring interpretation findings (“first interpretation findings” or “second interpretation findings”) created by a user based on the medical information, a comparison step for comparing the auto-generated findings with the interpretation findings (at least one of the “first interpretation findings” and the “second interpretation findings”), a presentation step for presenting to a user a result of comparison between the auto-generated findings and the interpretation findings (at least one of the “first interpretation findings” and the “second interpretation findings”) based on a “predetermined workflow”, and a setting step for setting the “predetermined workflow” based on a user operation.

In the present embodiment, when interpreting medical information (medical image in the present embodiment), analysis by computer processing (AI analysis) is performed in addition to the interpretation of the interpretation doctor, and the “auto-generated findings” that are the AI analysis result are compared with the interpretation findings (“first interpretation findings” or “second interpretation findings”) of the interpretation doctor. Therefore, it is possible to perform quality assurance (hereinafter, referred to as “QA”) for diagnostic accuracy regarding the medical information (medical image).

The first interpretation and the second interpretation are performed by different interpretation doctors (the first interpretation doctor and the second interpretation doctor). By switching whether or not to refer to the interpretation findings of the interpretation doctor (the first interpretation doctor and the second interpretation doctor), whether or not to refer to the analysis result (that is, “auto-generated findings”) by computer processing (AI analysis) at the time of each interpretation, it is possible to have various variations in the interpretation workflow.

Regarding which workflow is to be applied during interpretation, the hardware processor 31 as a setter sets a “predetermined workflow” in advance based on the user operation, and the set “predetermined workflow” is applied when interpretation is performed.

As for the “predetermined workflow”, for example, the user performs an input operation through the workflow setting screen 361 shown in FIGS. 4, 6, and 8 and operates a “register” button to transmit an operation signal corresponding to the user's input operation to the hardware processor 31, and the hardware processor 31 that receives the operation signal sets a “predetermined workflow” according to the user operation.

For example, when emphasis is placed on efficient interpretation, as shown in FIG. 4, first interpretation is performed by the doctor (first interpretation doctor) and AI analysis is performed by the hardware processor 31, and the “auto-generated findings” that are the AI analysis result is compared with the “first interpretation findings” that are the interpretation result of the first interpretation doctor. When the findings are + (with abnormal findings) in the AI analysis and − (no abnormal findings) in the first interpretation or when the findings are − (no abnormal findings) in the AI analysis and − (no abnormal findings) in the first interpretation, proceeding to the second interpretation of the second interpretation doctor (second interpreter) occurs, and the result of comparison between the “auto-generated findings” and the “first interpreting findings”, which are the interpretation result of the first interpretation doctor, is displayed in the worklist of the second interpretation doctor (second interpreter). Then, a flow in which the second interpretation doctor performs the second interpretation while referring to the comparison result displayed in the worklist or the interpretation report of the first interpretation doctor, which is the “first interpretation findings”, and sets the “second interpretation findings” as “definitive diagnostic information” is set as the “predetermined workflow”.

The frames showing the order of each interpretation and AI analysis shown in FIG. 4 can be rearranged as appropriate so that the user can achieve the desired order. It is also possible to appropriately rewrite what kind of display, processing, and the like are to be performed at each stage, that is, the specific content of “event”, conditions, and the like.

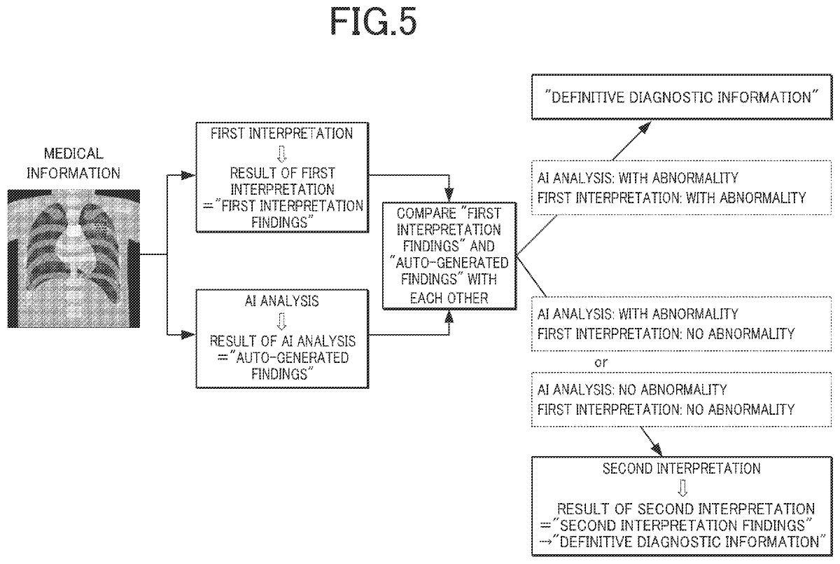

FIG. 5 is an explanatory diagram schematically showing the flow of interpretation processing when the flow shown in FIG. 4 is set as a “predetermined workflow”. The “predetermined workflow” shown in FIGS. 4 and 5 is assumed to be the “first workflow”.

As shown in FIG. 5, in this case, after obtaining the “first interpretation findings” by the interpretation of the first interpretation doctor and obtaining the “auto-generated findings” by computer processing (AI analysis), the hardware processor 31 as a comparer performs comparison processing between the “first interpretation findings” and the “auto-generated findings”.

When the result is + (with abnormal findings) in the AI analysis and − (no abnormal findings) in the first interpretation or when the result is − (no abnormal findings) in the AI analysis and − (no abnormal findings) in the first interpretation, proceeding to the second interpretation of the second interpretation doctor (second interpreter) occurs.

If the AI analysis result and the findings of the first interpretation doctor are different, it is necessary to check and determine which findings are correct in the second interpretation. Even if the AI analysis result matches the findings of the first interpretation doctor, when the findings are “− (no abnormal findings)”, it is preferable to check once again for any oversights in the second interpretation.

In these cases, as shown in FIG. 5, the second interpretation is checking interpretation and the “second interpretation findings”, which are the findings of the second interpretation doctor, are “definitive diagnostic information”.

When performing interpretation with such a workflow, if there is a doctor who performs checking interpretation to check each finding in addition to the first interpretation doctor and the second interpretation doctor, a checking interpretation doctor may be in charge of the “second interpretation” shown in FIG. 5.

The term “definitive diagnostic information” referred to herein is a diagnosis result determined and confirmed by the doctor (interpretation doctor) based on medical images and findings or analysis results obtained based thereon.

For the final diagnosis of a patient, for example, a clinician may make a determination by comprehensively taking into consideration examination data, medical examination data, and the like obtained from various examinations, medical examinations, and the like in addition to the determination of a doctor who creates the “definitive diagnostic information”. The term “definitive diagnostic information” hereinafter has the same meaning as described above.

On the other hand, in the case of + (with abnormal findings) in the AI analysis and + (with abnormal findings) in the first interpretation, “+ (with abnormal findings)” that is the matched findings is set as “definitive diagnostic information”, and the interpretation regarding the medical information ends.

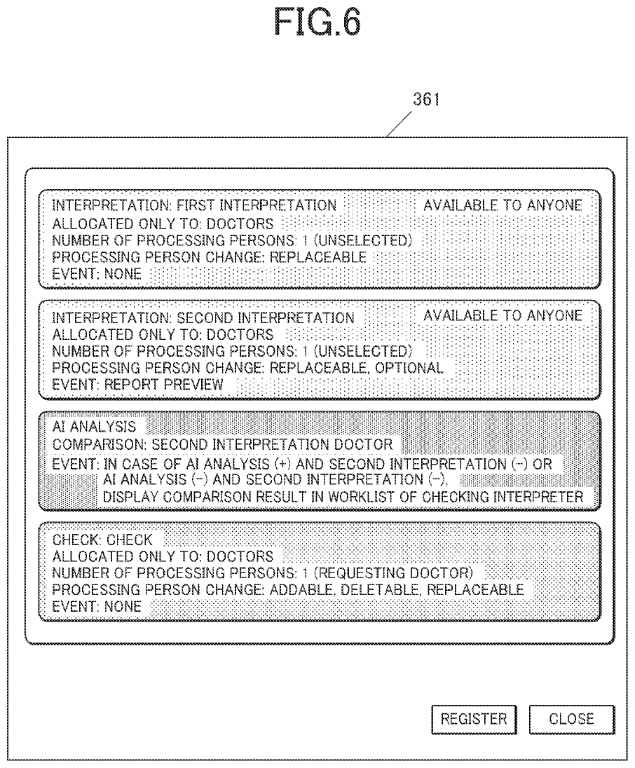

For example, when it is necessary to interpret images more carefully, as shown in FIG. 6, the first interpretation doctor performs first interpretation, and the second interpretation doctor performs second interpretation while referring to the “first interpretation findings”, such as the interpretation report obtained by the first interpretation, and separately from this, obtains “auto-generated findings” that are the result of AI analysis by the hardware processor 31. Then, the “auto-generated findings” that are the AI analysis result is compared with the “second interpretation findings” that are the interpretation result of the second interpretation doctor. When the findings are + (with abnormal findings) in the AI analysis and − (no abnormal findings) in the second interpretation or when the findings are − (no abnormal findings) in the AI analysis and − (no abnormal findings) in the second interpretation, proceeding to the checking interpretation of the checking interpretation doctor (checking interpreter) occurs, and the result of comparison between the “auto-generated findings” and the “second interpreting findings”, which are the interpretation result of the second interpretation doctor, is displayed in the worklist of the checking interpretation doctor (checking interpreter). Then, a flow in which the checking interpretation doctor performs the checking interpretation while referring to the comparison result displayed in the worklist, the interpretation report of the first interpretation doctor that is the “first interpretation findings”, or the interpretation report of the second interpretation doctor that is the “second interpretation findings”, and sets the findings of the checking interpretation doctor as “definitive diagnostic information” is set as the “predetermined workflow”.

The order of frames showing the order of each interpretation and AI analysis shown in FIG. 6, the specific content of “event”, conditions, and the like can be appropriately input by the user, which is the same as in the case shown in FIG. 4.

The checking interpretation doctor (checking interpreter) may be a doctor for checking interpretation who is different from the first interpretation doctor and the second interpretation doctor, or may be the second interpretation doctor.

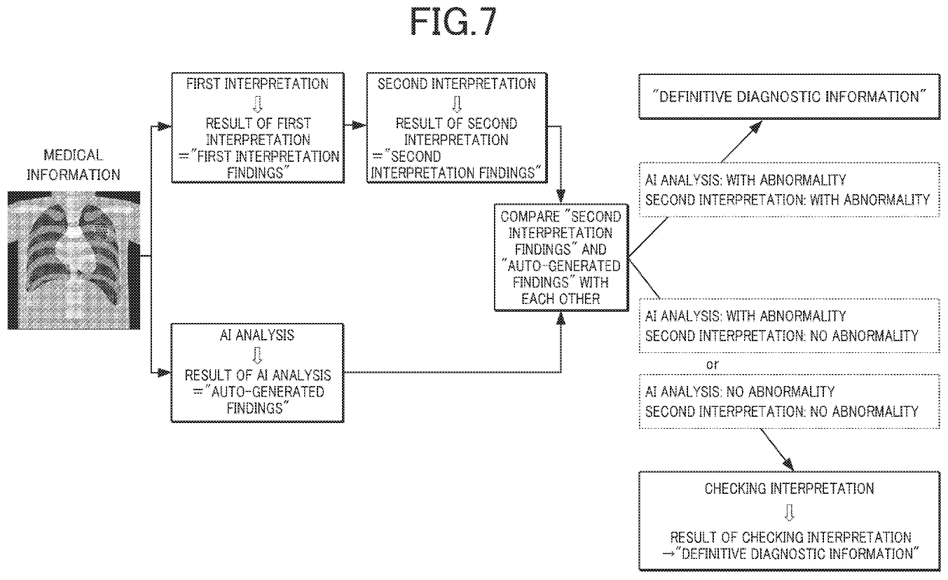

FIG. 7 is an explanatory diagram schematically showing the flow of interpretation processing when the flow shown in FIG. 6 is set as a “predetermined workflow”. The “predetermined workflow” shown in FIGS. 6 and 7 is assumed to be the “second workflow”.

As shown in FIG. 7, in this case, the first interpretation doctor obtains the “first interpretation findings”, and the second interpretation doctor obtains the “second interpretation findings” by performing the second interpretation while referring to the “first interpretation findings”. “Auto-generated findings” are acquired by computer processing (AI analysis). Thereafter, the hardware processor 31 as a comparer performs comparison processing between the “second interpretation findings” and the “auto-generated findings”.

When the result is + (with abnormal findings) in the AI analysis and − (no abnormal findings) in the first interpretation or when the result is − (no abnormal findings) in the AI analysis and − (no abnormal findings) in the first interpretation, proceeding to the checking interpretation of the checking interpretation doctor (checking interpreter) occurs.

If the AI analysis result and the findings of the second interpretation doctor are different, it is necessary to check and determine which findings are correct in the checking interpretation. Even if the AI analysis result matches the findings of the second interpretation doctor, when the findings are “− (no abnormal findings)”, it is preferable to check once again for any oversights in the checking interpretation.

In these cases, as shown in FIG. 7, the findings of the checking interpretation doctor are “definitive diagnostic information”.

On the other hand, in the case of findings of + (with abnormal findings) in the AI analysis and + (with abnormal findings) in the first interpretation, “+ (with abnormal findings)” that is the matched findings is set as “definitive diagnostic information”, and the interpretation regarding the medical information ends.

In the “first workflow”, which is the “predetermined workflow” shown in FIGS. 4 and 5, the second interpretation doctor performs the second interpretation as checking interpretation while referring to the “first interpretation findings” of the first interpretation doctor or the “auto-generated findings”, which are the AI analysis result, and the result of comparison between the “first interpretation findings” and the “auto-generated findings”.

For this reason, in the second interpretation, checking whether there are any oversights may be mainly performed focusing on the differences between the “first interpretation findings” and the “auto-generated findings”. Therefore, since the interpretation is efficiently performed, it can be expected to converge to “definitive diagnosis” at an early stage.

On the other hand, in the “first workflow”, since the second interpretation doctor performs interpretation after viewing the “first interpretation findings” of the first interpretation doctor or the “auto-generated findings”, there is a risk that the second interpretation will be biased to the “first interpretation findings” or the “auto-generated findings”.

In the “second workflow”, which is the “predetermined workflow” shown in FIGS. 6 and 7, the “auto-generated findings” are not referred to in the second interpretation. For this reason, the second interpretation is not influenced by the “auto-generated findings”, but the same is true in that the “first interpretation findings” of the first interpretation doctor are referred to. Therefore, there is still a risk that the second interpretation will be biased due to the influence of the “first interpretation findings”.

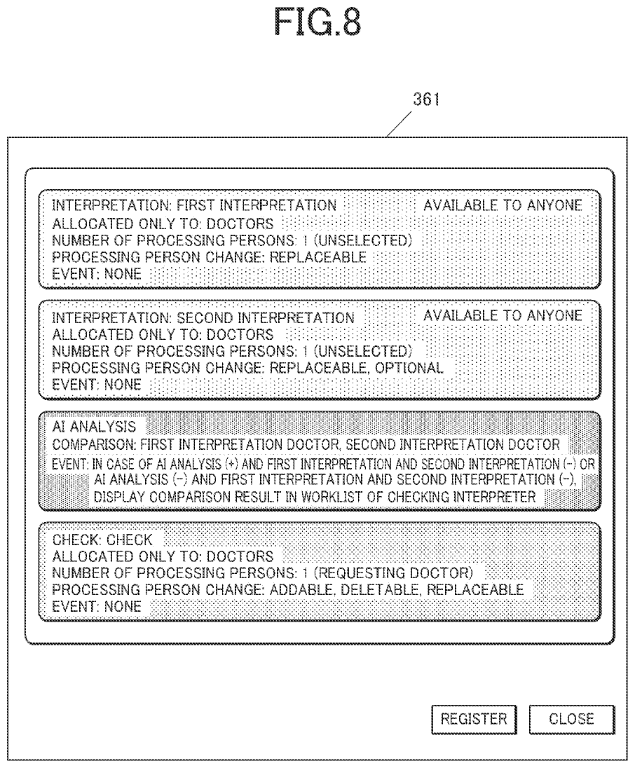

In this respect, for example, as shown in FIG. 8, first interpretation, second interpretation, and AI analysis are performed in parallel, and the “auto-generated findings” that are the AI analysis result, the “first interpretation findings” obtained by the first interpretation, and the “second interpretation findings” obtained by the second interpretation are compared with each other. If either the “first interpretation findings” or the “second interpretation findings” match the “auto-generated findings”, the matched findings are set as “definitive diagnostic information”. When such a flow is set as the “predetermined workflow”, the interpretation efficiency is somewhat sacrificed because the first interpretation and the second interpretation are performed without referring to anything, but since the first interpretation and the second interpretation are not influenced by other findings, more careful interpretation can be performed.

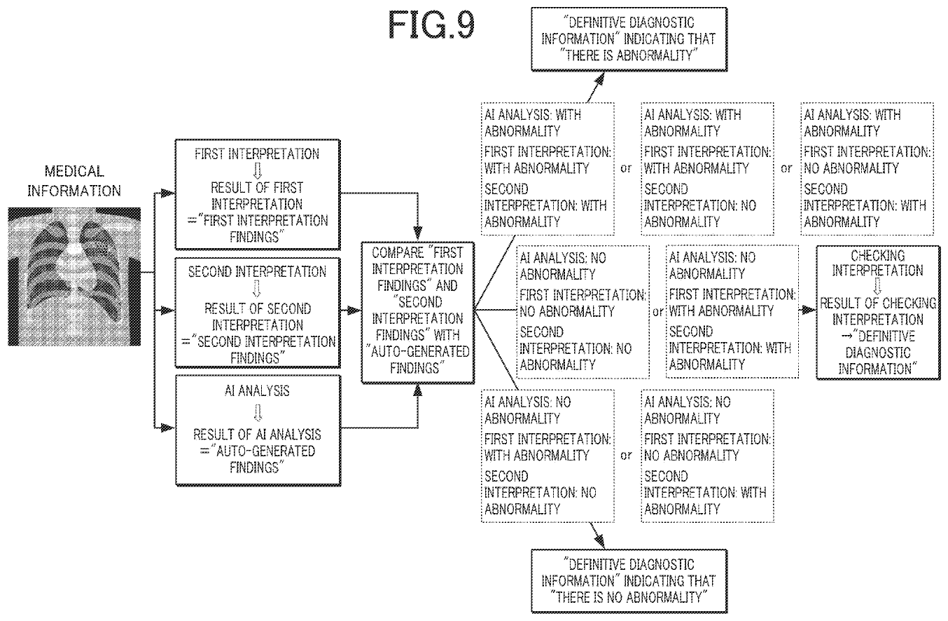

FIG. 9 is an explanatory diagram schematically showing the flow of interpretation processing when the flow shown in FIG. 8 is set as a “predetermined workflow”. The “predetermined workflow” shown in FIGS. 8 and 9 is assumed to be the “third workflow”.

As shown in FIG. 9, in this case, the first interpretation doctor obtains the “first interpretation findings” by performing the first interpretation, and the second interpretation doctor obtains the “second interpretation findings” by performing the second interpretation. “Auto-generated findings” are acquired by computer processing (AI analysis). Thereafter, the hardware processor 31 as a comparer performs comparison processing between the “first interpretation findings” and the “second interpretation findings” and the “auto-generated findings”.

As a result, when the “auto-generated findings” of + (with abnormal findings) are obtained by the AI analysis and the interpretation findings (“first interpretation findings” or “second interpretation findings”) of + (with abnormal findings) are obtained by at least one of the first interpretation and the second interpretation, the findings of + (with abnormal findings) that match the “auto-generated findings” are set as “definitive diagnostic information”.

When the “auto-generated findings” of − (no abnormal findings) are obtained by the AI analysis and the interpretation findings (“first interpretation findings” or “second interpretation findings”) of − (no abnormal findings) are obtained by at least one of the first interpretation and the second interpretation, the findings of − (no abnormal findings) that match the “auto-generated findings” are set as “definitive diagnostic information”.

That is, if the determination of the presence or absence of an abnormal part is different between a plurality of interpretation findings (“first interpretation findings” and “second interpretation findings”), interpretation findings matching the “auto-generated findings” that are the AI analysis result, among the plurality of interpretation findings, are adopted as correct findings, and the interpretation regarding the medical information ends.

For example, when the reliability of AI analysis is high, adopting such a workflow makes interpretation converge at an early stage, so that it is possible to perform efficient interpretation processing.

On the other hand, when the “auto-generated findings” of − (no abnormal findings) are obtained by the AI analysis and the interpretation findings (“first interpretation findings” and “second interpretation findings”) of − (no abnormal findings) are obtained by both the first interpretation and the second interpretation or when the “auto-generated findings” of − (no abnormal findings) are obtained by the AI analysis and the interpretation findings (“first interpretation findings” and “second interpretation findings”) of + (with abnormal findings) are obtained by both the first interpretation and the second interpretation, proceeding to the checking interpretation of the checking interpretation doctor (checking interpreter) occurs.

If the AI analysis result is different from the findings of the first interpretation doctor and the findings of the second interpretation doctor, it is necessary to check and determine which of the “auto-generated findings” and the interpreting findings (“first interpreting findings” and “second interpreting findings”) are correct in the checking interpretation. Even if the AI analysis result matches the findings of the first interpretation doctor and the findings of the second interpretation doctor, when the findings are “− (no abnormal findings)”, it is preferable to check once again for any oversights in the checking interpretation.

In these cases, as shown in FIG. 9, the findings of the checking interpretation doctor are “definitive diagnostic information”.

Each of the “first workflow,” the “second workflow,” and the “third workflow” exemplified above has advantages and disadvantages Depending on the level of proficiency of the first interpretation doctor and the second interpretation doctor, the accuracy level of AI analysis, and the situation of the facility where the interpretation management method is introduced, there is a difference in suitability. In this respect, in the present embodiment, the “predetermined workflow” adopted for interpretation management can be set based on the user operation. Therefore, it is possible to set the optimum workflow according to the point that the user desires to emphasize, the current facility situation, and the like.

The “predetermined workflow” that can be set by the hardware processor 31 as a setter is not limited to the “first workflow”, the “second workflow”, and the “third workflow” exemplified in the present embodiment.

For example, the user can set various conditions, such as in what case (that is, a pattern of combination of AI analysis (+) or (−), “first interpretation findings” (+) or (−), and “second interpretation findings” (+) or (−)) and what procedure (for example, whether or not to refer to the interpretation report, or whether or not to refer to the result of comparison with the AI analysis result) the interpretation is to be performed, on the workflow setting screen 361.

For example, in the “first workflow”, “second workflow”, and “third workflow”, even if the AI analysis result and the findings of the interpretation doctor (the first interpretation doctor and the second interpretation doctor) match each other, when the findings are “− (no abnormal findings)”, proceeding to the checking interpretation occurs to check again. However, a flow in which even with the findings of “− (no abnormal findings)”, if the AI analysis result and the findings of the interpretation doctor (the first interpretation doctor and the second interpretation doctor) match each other, proceeding to the checking interpretation is not made and the findings are set as “definitive diagnostic information” may be set as the “predetermined workflow”.

[Effect]

As described above, the analysis device 3 as an interpretation management device according to the present embodiment includes: a first acquirer that acquires “auto-generated findings” obtained by computer processing on medical information; a second acquirer that acquires “first interpretation findings” created by the user; a third acquirer that acquires “second interpretation findings” created by the user based on the medical information; the hardware processor 31 as a comparer that compares the “auto-generated findings” with at least one of the first interpretation findings and the second interpretation findings; the display 36 as a presenter that presents to the user a result of comparison between the “auto-generated findings” and the at least one interpretation findings (“first interpretation findings” or “second interpretation findings”) based on a “predetermined workflow”, and the hardware processor 31 as a setter that can set the “predetermined workflow” based on a user operation.

In this manner, it is possible to compare the result of computer processing (AI analysis) and the determination result of the user (doctor or interpretation doctor) during interpretation and present the comparison result to the user. Therefore, since the interpretation can be performed more carefully, it is possible to improve the accuracy and quality of interpretation.

In such a case, a “predetermined workflow” regarding in what order the users (doctors and interpretation doctors) should interpret images, what kind of information users (doctors and interpretation doctors) should refer to when performing interpretation, and the like can be set according to the user's request. Therefore, it is possible to realize a more preferable workflow setting according to the facility environment, the operational status of the facility, the differences in experience and preferences of users (doctors and interpretation doctors), and the like.

That is, since a desired interpretation workflow differs depending on the medical facility, it takes time and cost to develop and manufacture each device for realizing the desired workflow.

In this respect, in the present embodiment, in the interpretation diagnosis method for improving the interpretation quality by comparing the result of computer processing (AI analysis) with the determination result of the user (doctor or interpretation doctor) during interpretation, the timing of displaying the result of comparison between the AI analysis result and the doctor's interpretation result can be changed according to the user's request or the like on the interpretation doctor (the first interpretation doctor, the second interpretation doctor, or the like) side. Therefore, when it is necessary to perform efficient interpretation by focusing on differences in findings while referring to other findings or when it is necessary to perform careful interpretation without being affected by other findings, it is possible to customize the interpretation workflow appropriately according to the user's request.

In the present embodiment, the hardware processor 31 as a setter can set the “predetermined workflow” so as to be associated with information (for example, information regarding the presence or absence of an abnormal part) of the auto-generated findings and information (for example, information regarding the presence or absence of an abnormal part) of the at least one interpretation findings (“first interpretation findings” or “second interpretation findings”).

Therefore, depending on whether the “auto-generated findings” that are the result of computer processing (AI analysis) have abnormal findings or do not have abnormal findings, it is possible to change the “predetermined workflow” (order, content structure, and the like) during interpretation.

In other words, when the medical information (medical image) is determined to be “abnormal findings”, the medical information (medical image) should be examined more carefully by the clinician and the like who view the interpretation result. However, when the content of the determination by computer processing (AI analysis) is “no abnormal findings”, there is a possibility that the medical information (medical image) will not be examined more deeply. If there is an oversight in the AI analysis, there is also a possibility that the final determination will be erroneous. For this reason, when “no abnormal findings” are determined in the AI analysis, it is possible to adopt a workflow in which careful measures, such as performing further checking interpretation, are taken based on the determination.

In the present embodiment, when information indicating that there are abnormal findings is extracted from the “auto-generated findings” and information indicating that there are no abnormal findings is extracted from the at least one interpretation findings (“first interpretation findings” or “second interpretation findings”), the hardware processor 31 as a setter can set the “predetermined workflow” according to the user operation.

That is, when there is a difference in determination (findings) on medical information (medical image) between the “auto-generated findings” by computer processing (AI analysis) and the interpretation findings of the interpretation doctor (“first interpretation findings” or “second interpretation findings”), which findings should be adopted as correct findings, whether to perform second interpretation or checking interpretation, and the like can be set according to the user's request.

Therefore, it is possible to customize the interpretation workflow according to the facility situation, the reliability of AI analysis, the experience level of the interpretation doctor, and the like.

In the present embodiment, the hardware processor 31 as a comparer derives (calculates), as the comparison result, a result of comparison between information regarding the presence or absence of abnormal findings extracted from the “auto-generated findings” and information regarding the presence or absence of abnormal findings extracted from the at least one interpretation findings (“first interpretation findings” or “second interpretation findings”).

Therefore, it is possible to clarify whether or not the result of computer processing (AI analysis) and the interpretation result of the interpretation doctor match each other.

In the present embodiment, the display 36 as a presenter presents the “auto-generated findings” or the at least one interpretation findings (“first interpretation findings” or “second interpretation findings”) to the user based on the comparison result of the hardware processor 31 as a comparer.

Therefore, the result of computer processing (AI analysis), the interpretation result of the interpretation doctor, and the match or mismatch therebetween can be recognized by the user.

In the present embodiment, the hardware processor 31 as a setter can set whether or not to display the auto-generated findings or the at least one interpretation findings (“first interpretation findings” or “second interpretation findings”) so as to be associated with information (information regarding the presence or absence of abnormal findings) of the auto-generated findings and information (information regarding the presence or absence of abnormal findings) of the at least one interpretation findings (“first interpretation findings” or “second interpretation findings”).

For example, when it is determined that there are abnormal findings in the AI analysis, even if the interpretation doctor performs interpretation in a state in which the interpretation doctor is biased to the determination, the interpretation tends to be performed cautiously. For this reason, a serious error is unlikely to occur. On the other hand, when it is determined that there are no abnormal findings in the AI analysis, if the interpretation doctor performs interpretation after the determination result is displayed in advance, the interpretation tends to be performed with a bias that there will be no abnormality. For this reason, even if there is actually an abnormality, there is a risk of overlooking the abnormality.

Therefore, by adopting a workflow in which the display changes depending on whether or not there are abnormal findings, it is possible to perform more careful interpretation.

In the present embodiment, the hardware processor 31 as a setter can set, as the “predetermined workflow”, at least two workflows of a workflow in which the “second interpretation findings” are acquired after comparing the “auto-generated findings” with the “first interpretation findings” (“first workflow” in the present embodiment), a workflow in which the “second interpretation findings” are acquired after acquiring the “first interpretation findings” and the “auto-generated findings” and the “second interpretation findings” are compared with each other (“second workflow” in the present embodiment), and a workflow in which the “auto-generated findings” and the “first interpretation findings” and the “second interpretation findings” are compared with each other (“third workflow” in the present embodiment).

Therefore, in the interpretation, it is possible to adopt a workflow customized by the user according to the facility situation, the reliability of the AI analysis, the experience level of the interpretation doctor, and the like.

The analysis device 3 as an interpretation management device according to the present embodiment includes: a first acquirer that acquires “auto-generated findings” obtained by computer processing on medical information; a second acquirer that acquires “interpretation findings” created by a user (doctor or interpretation doctor) based on the medical information, the hardware processor 31 as a comparer that compares the “auto-generated findings” with the interpretation findings, the display 36 as a presenter that presents to a user a result of comparison between the auto-generated findings and the interpretation findings based on a predetermined workflow, and the hardware processor 31 as a setter that can set the predetermined workflow based on a user operation.

Thus, even if one interpretation findings of the interpretation doctor are acquired in addition to the “auto-generated findings” by AI analysis, the user can customize the workflow relevant to interpretation, such as whether or not to allow reference to “auto-generated findings” during interpretation, according to the facility situation, the reliability of AI analysis, the experience level of the interpretation doctor, and the like.

### Modification Examples

While the embodiment of the present invention has been described above, it is needless to say that the present invention is not limited to such an embodiment and various modifications can be made without departing from the scope of the present invention.

For example, in the embodiment described above, the case where the medical information to be analyzed by the analysis device 3 is a medical image is exemplified. However, the medical information is not limited to the medical “image”.

Information acquired by various examinations on patients may be widely included in medical information. For example, results obtained by various examinations, such as electrocardiogram waveform data, heart sound data, and blood flow data, may be included in the medical information. Even in the case of adopting a diagnosis flow in which AI analysis is performed on these pieces of medical information and the result of AI analysis is compared with the diagnosis result of a doctor, it is possible to build a flow that is more suitable for the operational status of the facility by applying the present invention.

In the present embodiment, in FIG. 1, the analysis device 3, the interpretation terminal 4, and the image server 5 are illustrated as separate and independent devices. However, the analysis device 3 and the image server 5 or the analysis device 3, the interpretation terminal 4, and the image server 5 may be configured as one device or one system.

In the present embodiment, the case is exemplified in which information regarding the presence or absence of abnormal findings is extracted from the “auto-generated findings” or the interpretation findings (“first interpretation findings” or “second interpretation findings”) and these are compared with each other. However, the information extracted from the “auto-generated findings” or the interpretation findings (“first interpretation findings” or “second interpretation findings”) is not limited to the information regarding the presence or absence of abnormal findings.

For example, the hardware processor 31 as a comparer may compare information regarding at least one of the type, number, and position of abnormal findings extracted from the “auto-generated findings” with information regarding at least one of the type, number, and position of abnormal findings extracted from at least one interpretation findings (that is, “first interpretation findings” or “second interpretation findings”) and derive (calculate) a result of the comparison.

In this case, in order to compare the two pieces of information with each other, the interpretation findings (“first interpretation findings” or “second interpretation findings”) are structured, and the structured pieces of data are compared with each other.

It is needless to say that the present invention is not limited to the above-described embodiments, modification examples, and the like and can be modified as appropriate without departing from the gist of the present invention.

Although embodiments of the present invention have been described and illustrated in detail, the disclosed embodiments are made for purposes of illustration and example only and not limitation. The scope of the present invention should be interpreted by terms of the appended claims

What is claimed is:

1. An interpretation management device, comprising:
a hardware processor,
wherein the hardware processor acquires auto-generated findings obtained by computer processing on medical information, acquires first interpretation findings created by a user based on the medical information, acquires second interpretation findings created by a user based on the medical information, compares the auto-generated findings with at least one of the first interpretation findings and the second interpretation findings, presents to a user a result of comparison between the auto-generated findings and the at least one interpretation findings based on a predetermined workflow, and allows the predetermined workflow to be set based on a user operation.

2. The interpretation management device according to claim 1,
wherein the hardware processor allows the predetermined workflow to be set so as to be associated with information of the auto-generated findings and information of the at least one interpretation findings.

3. The interpretation management device according to claim 2,
wherein, when information indicating that there are abnormal findings is extracted from the auto-generated findings and information indicating that there are no abnormal findings is extracted from the at least one interpretation findings, the hardware processor allows the predetermined workflow to be set according to a user operation.

4. The interpretation management device according to claim 1,
wherein the hardware processor derives, as the comparison result, a result of comparison between information regarding presence or absence of abnormal findings extracted from the auto-generated findings and information regarding presence or absence of abnormal findings extracted from the at least one interpretation findings.

5. The interpretation management device according to claim 1,
wherein the hardware processor derives, as the comparison result, a result of comparison between information regarding at least one of type, number, and position of abnormal findings extracted from the auto-generated findings and information regarding at least one of type, number, and position of abnormal findings extracted from the at least one interpretation findings.

6. The interpretation management device according to claim 1,
wherein the hardware processor presents the auto-generated findings or the at least one interpretation findings to the user based on the comparison result.

7. The interpretation management device according to claim 1,
wherein the hardware processor allows setting of whether or not to display the auto-generated findings or the at least one interpretation findings so as to be associated with information of the auto-generated findings and information of the at least one interpretation findings.

8. The interpretation management device according to claim 1,
wherein the hardware processor allows setting of, as the predetermined workflow, at least two workflows of a workflow in which the second interpretation findings are acquired after comparing the auto-generated findings with the first interpretation findings, a workflow in which the second interpretation findings are acquired after acquiring the first interpretation findings and the auto-generated findings and the second interpretation findings are compared with each other, and a workflow in which the auto-generated findings and the first interpretation findings and the second interpretation findings are compared with each other.

9. An interpretation management device, comprising:
a hardware processor,
wherein the hardware processor acquires auto-generated findings obtained by computer processing on medical information, acquires interpretation findings created by a user based on the medical information, compares the auto-generated findings with the interpretation findings, presents to a user a result of comparison between the auto-generated findings and the interpretation findings based on a predetermined workflow, and allows the predetermined workflow to be set based on a user operation.

10. A non-transitory recording medium storing a computer readable program causing a computer to perform:
acquiring auto-generated findings obtained by computer processing on medical information;
acquiring first interpretation findings created by a user based on the medical information;
acquiring second interpretation findings created by a user based on the medical information;
comparing the auto-generated findings with at least one of the first interpretation findings and the second interpretation findings;
presenting to a user a result of comparison between the auto-generated findings and the at least one interpretation findings based on a predetermined workflow; and
allowing setting of the predetermined workflow based on a user operation.

11. The non-transitory recording medium storing a computer readable program according to claim 10,
wherein, in the setting, the predetermined workflow can be set so as to be associated with information of the auto-generated findings and information of the at least one interpretation findings.

12. The non-transitory recording medium storing a computer readable program according to claim 11,
wherein, when information indicating that there are abnormal findings is extracted from the auto-generated findings and information indicating that there are no abnormal findings is extracted from the at least one interpretation findings, in the setting, the predetermined workflow can be set according to a user operation.

13. The non-transitory recording medium storing a computer readable program according to claim 10,
wherein, in the comparison, a result of comparison between information regarding presence or absence of abnormal findings extracted from the auto-generated findings and information regarding presence or absence of abnormal findings extracted from the at least one interpretation findings is derived as the comparison result.

14. The non-transitory recording medium storing a computer readable program according to claim 10,
wherein, in the comparison, a result of comparison between information regarding at least one of type, number, and position of abnormal findings extracted from the auto-generated findings and information regarding at least one of type, number, and position of abnormal findings extracted from the at least one interpretation findings is derived as the comparison result.

15. The non-transitory recording medium storing a computer readable program according to claim 10,
wherein, in the presentation, the auto-generated findings or the at least one interpretation findings are presented to the user based on the comparison result in the comparison.

16. The non-transitory recording medium storing a computer readable program according to claim 10,
wherein, in the setting, whether or not to display the auto-generated findings or the at least one interpretation findings can be set so as to be associated with information of the auto-generated findings and information of the at least one interpretation findings.

17. The non-transitory recording medium storing a computer readable program according to claim 10,
wherein, in the setting, at least two workflows of a workflow in which the second interpretation findings are acquired after comparing the auto-generated findings with the first interpretation findings, a workflow in which the second interpretation findings are acquired after acquiring the first interpretation findings and the auto-generated findings and the second interpretation findings are compared with each other, and a workflow in which the auto-generated findings and the first interpretation findings and the second interpretation findings are compared with each other can be set as the predetermined workflow.

18. A non-transitory recording medium storing a computer readable program causing a computer to perform:
acquiring auto-generated findings obtained by computer processing on medical information;
acquiring interpretation findings created by a user based on the medical information;
comparing the auto-generated findings with the interpretation findings;
presenting to a user a result of comparison between the auto-generated findings and the interpretation findings based on a predetermined workflow; and
allowing setting of the predetermined workflow based on a user operation.

19. An interpretation management method, comprising:
acquiring auto-generated findings obtained by computer processing on medical information;
acquiring first interpretation findings created by a user based on the medical information;
acquiring second interpretation findings created by a user based on the medical information;
comparing the auto-generated findings with at least one of the first interpretation findings and the second interpretation findings;
presenting to a user a result of comparison between the auto-generated findings and the at least one interpretation findings based on a predetermined workflow; and
allowing setting of the predetermined workflow based on a user operation.
